(19) United States
(12) Patent Application Publication
(10) Pub. No.: US 2009/0130130 A1
(43) Pub. Date: May 21, 2009

(54) MUTANT ALLERGEN(S)

(75) Inventors: Kaw Yan Chua, Singapore (SG); Fong Cheng Yi, Singapore (SG)

Correspondence Address: VOLPE AND KOENIG, P.C., UNITED PLAZA, SUITE 1600, 30 SOUTH 17TH STREET, PHILADELPHIA, PA, 19103, US

(73) Assignee: NATIONAL UNIVERSITY OF SINGAPORE, Singapore (SG)

(21) Appl. No.: 11/916,895

(22) PCT Filed: Jun. 9, 2006
(86) PCT No.: PCT/SG2006/000150

§ 371 Date: Dec. 7, 2007

Related U.S. Application Data
(60) Provisional application No. 60/689,172, filed on Jun. 10, 2005.

(51) Int. Cl. A61K 39/35; C07K 14/00; C12N 15/11; C12N 1/21; G01N 33/566; C07K 16/18; C12P 21/04; A61K 38/16

(52) U.S. Cl. 424/185.1; 530/350; 536/23.7; 435/320.1; 435/252.33; 435/69.1; 514/12; 530/387.9; 436/501

(57) ABSTRACT

There is provided an isolated polypeptide, derivative, isoform and/or fragment thereof, wherein the polypeptide is a mutant of a Group 5 allergen. There are also provided pharmaceutical compositions(s) and vaccine(s) for the treatment of subjects allergic to at least one Group 5 allergen.

### FIELD OF THE INVENTION

The present invention is in the field of molecular biology and immunology. In particular, the present invention relates to novel mutant allergen(s). More particularly, the present invention relates to Group 5 mutant allergens.

### BACKGROUND OF THE INVENTION

Type I allergy affects more than 25% of the world's population (Miyamoto, 1992), especially in industrialized countries. Allergic patients are characterized by their hereditary tendency to produce large amounts of IgE antibody to common environmental allergens. Cross-linking of effector cell-bound IgE antibodies by intact allergens leads to the release of biologically active mediators (histamines, leukotriences) which cause the immediate symptoms of type I allergy. IgE-mediated allergen presentation to T cells greatly enhances T cell activation, release of Type 2 proinflammatory cytokines, and thus late reactions.

Specific allergen immunotherapy (SIT) is an effective prophylactic treatment for atopic IgE-mediated disease, in particular for severe seasonal allergic rhinitis (Mailing, 1998; Bousquet et al. 1998; Durham et al. 1999; Walker et al. 2001). SIT has been used for more than 90 years for the management of allergic disorders, including seasonal and perennial allergic rhinitis, allergic asthma, and hymenoptera sensitivity.

The efficacy of specific allergen immunotherapy (SIT) in selected patients with IgE-mediated disease has led to interest in the mechanism underlying this treatment. There were a variety of immunological changes reported following immunotherapy. These include a moderate reduction of allergen specific IgE in serum (Van Ree et al. 1997) and an increase in allergen-specific IgG antibodies, particularly of IgG4 isotype (Devey et al. 1976; Aalberse et al. 1983; Gehlhar et al. 1999), reduction of effector cells (mast cells and eosinophils) at allergic mucosa sites (Wilson et al. 2001), reduction in basophils reactivity to allergen (Kimura et al. 1985), suppression of allergen-induced mediators secretion (Creticos et al. 1985), and induction of IL-10-producing T cells, which might be regulatory T cells.

The quantity of IL-4 produced by allergen specific memory CD4+ T cells from allergic subjects reduced considerably with treatment by allergen immunotherapy. Immunotherapy reduced IL-4 production by allergen specific CD4+ T cells to levels similar to non-allergic subjects, or to levels induced with non-allergic antigens such as tetanus toxoid (Secrist et al. 1993).

However, it is now clear that the induction of peripheral tolerance in antigen-specific T cells and increased production of IL-10 and/or TGF-b is essential for successful SIT (Akdis et al. 1998; Akdis and Blaser, 1999). In humans, IL-10 poses numerous potential anti-allergic properties that might be crucial for immunotherapy. These include inhibition of IgE-dependent mast cell activation (Royer et al. 2001), and inhibition of human eosinophil cytokine production and survival (Takanaski et al. 1994).

In T cells, IL-10 suppresses production of pro-allergic cytokines, such as IL-5 (Francis et al. 2003), and is able to induce a state of antigen-specific hyporesponsiveness or anergy (Groux et al. 1997).

IL-10 is a T cell derived cytokine that down-regulates both Th1 and Th2-type responses and appears to suppress both IgE-mediated inflammation and delayed-type hypersensitivity inflammation (de Waal et al. 1991; Fiorentino et al. 1991; Del Prete et al. 1993).

Together with IFN-gamma, IL-10 can also decrease the release of histamine and other mediators from mast cells and basophils (Royer et al. 2001; Pierkes et al. 1999). IL-10 is a potent suppressor of both total- and allergen-specific IgE, while favouring B cell switching to IgG4 in the presence of IL-4 (Akdis et al. 1998; Jeannin et al. 1998; Punnonen et al. 1993).

IL-10 has been regarded as the main cytokine in the peripheral tolerance observed in venom, pollen and house dust mite immunotherapy (Akdis et al. 1998; Bellinghausen et al. 1997; Savolainen et al. 2004; Jutel et al. 2003; Gardner et al. 2004). IL-10-producing cells have been detected in both the peripheral blood and nasal mucosa after immunotherapy (Nouri-Aria et al. 2004).

Subjects suffering from allergic rhinitis successfully treated with pollen SIT (good outcome) have higher levels of IL-10 mRNA in their allergen-stimulated T cells than subjects treated in the same way, but had poor or moderate outcome. The study also suggested that successful SIT depends on fast development and accessibility of IL-10 secreting T cells (Jutel et al. 2003). A report of house dust mite-SIT (HDM-SIT) in patients with house dust mite allergy demonstrated an increase in intracellular IL-10 production in CD4+CD25+ T lymphocytes after 70 days of treatment (Gardner et al. 2004).

SIT involves the administration of incremental doses of allergen into sensitized subjects in order to achieve a state of clinical tolerance to subsequent exposure (Rolland and O'Hehir, 1998). SIT using subcutaneous injections of an increasing dose of allergen have been shown to be effective in children and adults suffering from allergic rhinitis and asthma (Abramson et al. 2000).

One major problem with SIT is the concern that the administration of allergenic material may cause severe, life-threatening anaphylactic reactions (Mailing, 1998; Malling and Weeke, 1993; Bousquet et al. 1998). A number of strategies aim to improve the safety and efficacy of allergen-specific immunotherapy for allergic disease by selectively attenuating the allergenicity of the allergen, leaving immunogenicity and antigenicity unaffected. In other words, reducing unfavourable IgE-mediated side effects (e.g. histamine production) while maintaining immunogenic activity (e.g. stimulating T-cells).

Prior studies of major pollen allergens Cor a I and Bet v 1 showed that their isoforms possess different antigenic and allergenic properties, mainly due to a few but significant changes in their amino acid sequences (Ferreira et al. 1996; Breiteneder et al. 1993). Deletion mutants of Phl p 5—the major timothy grass allergen—have been created and shown to have reduced IgE binding capacity. These deletions were made outside the dominant T-cell region (Schramm et al. 1999).

Swoboda et al. showed that few point mutations in the sequence of Lol p 5 (the major ryegrass pollen allergen) produce mutants with wild-type-like structure but reduced allergenic activity.

The C8-C119 mutant of Der f 2 (mite allergen) was shown to give rise to both a markedly reduced skin prick test (SPT) (Kusonuki et al. 2000; Takai et al. 1997) and in vitro histamine release, but retained a completely intact capacity to stimulate T-cells (Takai et al. 1997). In addition, it was found that this mutant allergen almost exclusively induced the differentiation of Th1 cells from peripheral blood mononuclear cells (PBMC) of atopic individuals; and this effect could not be accounted for by a high allergen concentration, because high-dose tolerance detected by Der f 2 was not observed by the same concentration of C8/119S. Thus, the exclusive induction of Th1 cell differentiation was unlikely due to the shift of Ag dosage effect on the Th1/Th2 differentiation profile (Korematsu et al. 2000).

It has been reported that the T-cell epitope-containing fragments of Bet v 1 exhibited a more than 100-fold reduction in allergenic activity and were able to induce antibodies in vivo, which blocked patients' IgE binding to the wild-type allergen (Vrtala et al. 2000). Study of cow dander allergen also showed that when two overlapping recombinant fragments of Bos d 2 (corresponding to amino acids 1-131 and 81-172, respectively) covering the whole molecule were compared with the complete recombinant Bos d 2, only a low level of IgE reactivity was observed (Zeiler et al. 1997). These modified molecules represent useful candidates for more efficient specific immunotherapy.

Recently, a stable recombinant oligomer of Bet v 1 molecule that preserves secondary structure element, B cell and T cell epitopes of the wild-type allergen has been shown to exhibit reduced allergenic activity in clinical skin test studies of Swedish and French population (van-Hage Hamsten et al. 1999; Pauli et al. 2000). Vaccination with this genetically engineered hypoallergenic Bet v 1 allergen has been carried out in a multi-centre study, where 124 birch pollen-allergic patients were recruited. The study showed that active treatment with this molecule was able to induce protective immunoglobin G (Ig G) antibodies against new epitopes and a mixed Th2/Th1-like immune response (Neiderberger, 2004).

Allergy has been classified by the World Health Organization (WHO) as the fourth most important disease in the world. The WHO has estimated that 40% to 50% of the world's population could be affected by allergies by 2010, and it is estimated that house dust mites account for 50% of all cases of allergy, therefore, it is a major source of allergens that trigger asthma worldwide. The prevalence of asthma varies greatly between 3% and 30% of total population, and it is found to be more common in countries in the tropics and sub-tropics. Epidemiological studies revealed that Blo t 5 is one of the most important mite allergens worldwide. Up to 70% of the asthmatic and rhinitis patients in the tropics and sub-tropics had high IgE titres to Blo t 5 indicating that sensitization by Blo t 5 is the major trigger for asthma and rhinitis in these regions (Arruda et al. 1995; Kuo et al. 2003).

However, using wild type Blo t 5 allergens for immunotherapy has the risk of causing allergenic reactions in allergic subjects. Accordingly, there is a need in this field of medicine for novel hypoallergenic allergens.

### SUMMARY OF THE INVENTION

The present invention addresses the problems above, and provides new and/or improved mutant allergen(s). In particular, the present invention provides hypoallergenic mutants of Group 5 allergens.

Accordingly, the present invention provides an isolated polypeptide, derivative and/or fragment thereof, selected from the group consisting of:
(a) a polypeptide, derivative, isoform and/or fragment thereof, comprising a non-helical mutant of at least one Group 5 allergen;
(b) a polypeptide, derivative, isoform and/or fragment thereof, comprising an amino acid sequence corresponding to SEQ ID NO:33;
(c) a polypeptide, derivative, isoform and/or fragment thereof which exhibits reduced IgE reactivity compared to the wild type Group 5 allergen in subjects allergic to at least one Group 5 allergen;
(d) a polypeptide, derivative, isoform and/or fragment thereof which exhibits reduced skin reactivity compared to the wild type Group 5 allergen in subjects allergic to at least one Group 5 allergen;
(e) a polypeptide, derivative, isoform and/or fragment thereof which reduces histamine release compared to the wild type Group 5 allergen in subjects allergic to at least one Group 5 allergen;
(f) a polypeptide, derivative, isoform and/or fragment thereof which induces increased IFN-gamma production compared to the wild type Group 5 allergen in subjects allergic to at least one Group 5 allergen;
(g) a polypeptide, derivative, isoform and/or fragment thereof which induces increased IL-10 production compared to the wild type Group 5 allergen in subjects allergic and/or non-allergic to at least one Group 5 allergen;
(h) the polypeptide, derivative, isoform and/or fragment thereof of (a), (b), (c), (d), (e), (f) and/or (g) comprising at least one amino acid substitution, addition, deletion, and/or at least one chemical modification, and wherein the polypeptide exhibits the same or further reduced IgE reactivity than that of the polypeptide of (a), (b), (c), (d), (e), (f) and/or (g), in subjects allergic to at least one Group 5 allergen; and
(i) a polypeptide, derivative, isoform and/or fragment thereof, wherein the polypeptide comprises an amino acid sequence of at least 60% homology with SEQ ID NO:33.

The Group 5 allergen may be selected from the group consisting of Blo t 5, Der p 5, Der f 5, Der m 5. In particular, the Group 5 allergen is Blo t 5 allergen. In particular, the polypeptide according to the invention is a polypeptide comprising at least one of the amino acid substitutions corresponding to Leu57, Arg58, Arg74, Glu75, Glu79, Lys92, Lys94, Glu98, Gln102, Asp105, Lys106 of SEQ ID NO:33.

The polypeptide according to the invention may be a Group 5 allergen is from mite. In particular, the mite is house dust and/or storage mite. The Group 5 allergen may be from at least one organism selected from the group consisting of the Blomia genus, the Dermatophagoides genus, the Euoglyphus genus, the Glycyphagus genus, the Lepidoglyphus genus, the Acarus genus and/or the Tyrophagus genus. In particular, the Group 5 allergen is from Blomia tropicalis.

In particular, the isolated polypeptide, derivative, isoform and/or fragment thereof, according to the invention may be at least one polypeptide, derivative, isoform and/or fragment thereof comprising at least one amino acid sequence selected from the group consisting of SEQ ID NO:17, SEQ ID NO:19, SEQ ID NO:21, SEQ ID NO:23, SEQ ID NO: 25, SEQ ID NO:27, SEQ ID NO:29, SEQ ID NO:31, and/or SEQ ID NO:33.

There is also provided an isolated nucleic acid molecule encoding at least one polypeptide according to the invention. In particular, the isolated nucleic acid molecule is selected from the group consisting of SEQ ID NO:16, SEQ ID NO:18, SEQ ID NO:20, SEQ ID NO:22, SEQ ID NO:24, SEQ ID NO:26, SEQ ID NO:28, SEQ ID NO:30, and/or SEQ ID NO:32.

There is also provided a vector comprising at least one nucleic acid molecule according to the invention. There is also provided a host cell comprising at least one nucleic acid molecule according to the invention and/or at least one vector according to the invention. The host cell may be a prokaryotic and/or eukaryotic cell.

There is also provided a method for producing any polypeptide according to the invention. In particular, the method comprises the step of culturing the host cell according to the invention in a medium and isolating the polypeptide from the culture.

There is also provided an isolated oligonucleotide comprising at least one nucleic acid sequences selected from the group consisting of SEQ ID NO:1 to SEQ ID NO:15.

There is also provided a pharmaceutical composition comprising an effective amount of at least one polypeptide according to the invention. The pharmaceutical composition may further comprise at least one pharmaceutically acceptable carrier, diluent, adjuvant, excipients, or a combination thereof. The pharmaceutical composition may be for local, subcutaneous, intravenal, topical to a tissue locus, parenteral and/or oral administration.

There is also provided a vaccine against allergic reactions elicited by a naturally occurring allergen in patients suffering from allergy, comprising at least one polypeptide according to the invention. The vaccine may further comprise at least one adjuvant. The adjuvant may be any suitable therapeutically effective adjuvant.

There is also provided a process for preparing a pharmaceutical composition according to the invention comprising mixing at least one polypeptide according to the invention with at least one pharmaceutically acceptable substance and/or excipients.

There is also provided at least one polypeptide according to the invention for use in medicine. There is also provided at least one polypeptide for the preparation of a medicament for the prevention and/or treatment of allergy. In particular, the allergy may be Type I allergy. There is also provided the use of at least one polypeptide according to the invention for the preparation of a medicament for specific allergen immunotherapy (SIT).

There is also provided an isolated antibody, wherein the antibody specifically binds to at least one polypeptide according to the invention.

There is also provided a kit comprising at least one polypeptide according to the invention. There is also provided a kit comprising at least one pharmaceutical composition and/or a vaccine according to the invention. The kit may be an immunotherapeutic kit.

There is also provided a diagnostic assay for assessing relevance, safety or outcome of therapy of a subject using at least one polypeptide according to the invention and/or at least one antibody according to the invention, wherein at least one IgE containing sample of the subject is mixed with the polypeptide and/or antibody and assessed for the level of IgE reactivity.

### BRIEF DESCRIPTION OF THE FIGURES

FIG. 1. In vitro IgE reactivity of Blo t 5 allergen recombinant mutants in thirty Blo t 5-positive (allergic) subjects. Graph A: IgE reactivity of M1 to M4 as compared to Blo t 5. Graph B: IgE reactivity of M6 to M9 as compared to Blo t 5 (p<0.02).

FIG. 2. Human IgE inhibition assay of fifteen Blo t 5-positive atopic (allergic) subjects. Reduction in percentage of IgE inhibition for M9 (mutant protein) showed that the IgE binding epitopes in M9 have been disrupted.

Legend: Without inhibitor: IgE binding directly to the Blo t 5; Self: Blo t 5 had been added to the IgE prior to reaction to Blo t 5 on ELISA; CM: mutant M9, added to react to IgE prior to the reaction to Blo t 5 on ELISA; GST: control unrelated protein added to IgE prior to addition to ELISA.

FIG. 3. Circular dichroism analysis of Blo t 5 vs. M9 at 25° C. showed that the secondary structure of mutant protein M9 has lost the alpha-helical structure of the wild type protein, Blo t 5.

FIG. 4(A, B AND C). The comparative assessment of the allergenicity of recombinant mutant allergen M9 and native Blo t 5 based on the wheal sizes by skin prick tests on 38 B. tropicalis skin test positive (atopic) subjects. The results are shown in a graph for the diameter of the skin reaction in millimetres against the wild type and mutant allergen (graph A). Reduction in skin reactivity to M9 as compared to Blo t 5 on two representative individuals, the M9 wheal sizes being smaller (photo B and C).

FIG. 5(A to F). Histamine release on challenge with different concentrations of Blo t 5(∘) and M9 (▪) in 7 Blo t 5 allergic subjects. Percentage total histamine release (y-axis) against concentration of Blo t 5 and M9 (x-axis) in allergic subjects. The results are expressed as a percentage of total histamine release.

FIG. 6. Histamine release on challenge with different concentrations of Blo t 5(∘) and M9 (▪) in 1 Blo t 5 non-allergic subject. Percentage total histamine release (y-axis) against concentration of Blo t 5 and M9 (x-axis) in non-allergic subjects. The results are expressed as a percentage of total histamine release. No histamine was released in the non-allergic subject.

FIG. 7(A to F). Increase in IFN-gamma production by peripheral blood mononuclear cells (PBMC) of five allergic subjects (graph A to E) cultured with M9 as compared to Blo t 5. Non-allergic subject (graph F) showed no difference in IFN-gamma production. No detectable IL-4 produced in PBMC when cultured with both proteins.

FIG. 8(A to F). Increase in IL-10 production by peripheral blood mononuclear cells (PBMC) cultured with M9 as compared to Blo t 5 in all subjects (graph A to E allergic subjects, graph F non-allergic subject).

FIG. 9. Nucleotide and amino acid sequences of mutants M1 to M3. Mutated points are shown in bold italic.

FIG. 10. Nucleotide and amino acid sequences of mutants M4 to M6. Mutated points are shown in bold italic.

FIG. 11. Nucleotide and amino acid sequences of mutants M7 to M9. Mutated points are shown in bold italic.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present invention provides new and/or improved mutant allergen(s). In particular, the present invention provides hypoallergenic mutants of Group 5 allergens.

Accordingly, the present invention provides an isolated polypeptide, derivative and/or fragment thereof, selected from the group consisting of:
(a) a polypeptide, derivative, isoform and/or fragment thereof, comprising a non-helical mutant of at least one Group 5 allergen;
(b) a polypeptide, derivative, isoform and/or fragment thereof, comprising an amino acid sequence corresponding to SEQ ID NO:33;
(c) a polypeptide, derivative, isoform and/or fragment thereof which exhibits reduced IgE reactivity compared to the wild type Group 5 allergen in subjects allergic to at least one Group 5 allergen;
(d) a polypeptide, derivative, isoform and/or fragment thereof which exhibits reduced skin reactivity compared to the wild type Group 5 allergen in subjects allergic to at least one Group 5 allergen;
(e) a polypeptide, derivative, isoform and/or fragment thereof which reduces histamine release compared to the wild type Group 5 allergen in subjects allergic to at least one Group 5 allergen;
(f) a polypeptide, derivative, isoform and/or fragment thereof which induces increased IFN-gamma production compared to the wild type Group 5 allergen in subjects allergic to at least one Group 5 allergen.
(g) a polypeptide, derivative, isoform and/or fragment thereof which induces increased IL-10 production compared to the wild type Group 5 allergen in subjects allergic and/or non-allergic to at least one Group 5 allergen;
(h) the polypeptide, derivative, isoform and/or fragment thereof of (a), (b), (c), (d), (e), (f) and/or (g) comprising at least one amino acid substitution, addition, deletion, and/or at least one chemical modification, and wherein the polypeptide exhibits the same or further reduced IgE reactivity than that of the polypeptide of (a), (b), (c), (d), (e), (f) and/or (g), in subjects allergic to at least one Group 5 allergen;
(i) a polypeptide, derivative, isoform and/or fragment thereof, wherein the polypeptide comprises an amino acid sequence of at least 60% homology with SEQ ID NO:33.

The isolated polypeptide, derivative and/or fragment of (a) to (i) may be defined as a polypeptide with an amino acid sequence substantially identical with the polypeptide, with conserved amino acid changes that can be made without altering the function of the polypeptide according to the invention.

A non-helical polypeptide of (a) to (i) refers to a polypeptide wherein the predominant secondary structure is not alpha-helical and/or the predominant tertiary structure is not helical coiled coil. A non-helical polypeptide may be further defined as linear, and/or the circular diachroism trace of a non-helical polypeptide may have minimum ellipticities between the wavelengths of 200 to 240 nm. It will be obvious to a skilled person how to determine the degree of helicity of a polypeptide, by measuring the circular dichroism molar ellipticities of the polypeptide.

A mutant may be defined as comprising an amino acid sequence with at least one amino acid substitution, addition and/or deletion from the wild type amino acid sequence.

An allergen may be defined as a protein, naturally occurring wild type, recombinant and/or mutant. The present invention is addressed to mutant allergen(s).

‘Allergic’ may be defined as a term that may be used interchangeably with ‘atopic’. An allergic (atopic) subject may be defined as a person who is hypersensitised to allergens and in particular, the person experiences allergic reactions e.g. anaphylaxis upon exposure to the allergens. In particular, an allergic reaction may be caused by the subject having a Type I allergy.

A Group 5 allergen may be defined as a group of allergens determined by the World Health Organisation/International Union of Immunological Societies (WHO/IUIS) Allergen Nomenclature. In particular, a Group 5 allergen may be defined as encompassing the allergens already discovered and/or additions to the ‘Group 5’ class of allergens and is further defined as comprising all naturally occurring and/or recombinant derivative, isoforms, and/or fragments of a Group 5 allergen for all particular species.

IgE is well known to the skilled person, and may be defined as ‘immunoglobulin E’. In particular, IgE may be defined as human IgE.

IgE reactivity may be defined as the in vitro measurement of the degree and/or extent of interaction between IgE and the polypeptide according to the invention. In particular, the measurement is compared to the degree and/or extent of interaction between the wild type allergen and IgE. The method of measurement of IgE reactivity may be defined in particular as the method of using an ELISA IgE immunoassay and/or any other appropriate quantitative methods known in the art.

According to the present invention, the expression “reduced IgE reactivity as compared to the wild type Group 5 allergen” may be defined to mean that the reduction in IgE reactivity is measurable in a statistically significant manner (p<0.05) in at least one immunoassay using serum from a subject allergic to the wild type allergen. Preferably, the IgE reactivity is reduced by at least 5%, more preferably by at least 10%.

Skin reactivity may be defined as the in vivo appearance of wheals on the surface of animal skin, preferably in humans. Reduced skin reactivity of the polypeptide according to the invention compared to the wild type allergen may be defined as a reduction in diameter and/or area of visible wheals on the skin as observed when the in vivo skin prick test (SPT) is used, a method well known to a skilled person in the art.

Histamine may be defined as 2-(4-imidazolyl)ethylamine, and is a biogenic amine chemical well known to a skilled person.

Reduced histamine release may be defined as the reduced release of histamine from basophilic granulocytes upon stimulation by the polypeptide according to the invention compared to stimulation by the wild type allergen. Basophilic granulocytes may be defined in particular as human basophilic granulocytes. The quantitative in vitro detection of the decrease in total percentage of histamine may be measured using any appropriate immunoassay, a method well known to a person skilled in the art.

IFN-gamma may be defined as ‘interferon-gamma’ and is a cytokine well known to a skilled person. IFN-gamma may be defined in particular as human IFN-gamma.

The increased production of IFN-gamma may be defined as the increased production of IFN-gamma by T-cells stimulated by the polypeptide according to the invention as compared to the wild type allergen; this increase may be measured with a T-cell cytokine assay, the method being well known to a person skilled in the art.

IL-10 may be defined as ‘interleukin-10’ and is a cytokine well known to a skilled person in the art. Increased production of IL-10 by the polypeptide according to the invention compared to the wild type allergen may be defined as being measured with a T-cell cytokine assay, the method being well known to a skilled person.

Same IgE reactivity may be defined as this polypeptide (h) according to the invention having the same reduction in the measured degree and/or extent of interaction of IgE as compared to the polypeptide (a), (b), (c), (d), (e), (f) and/or (g), according to the invention.

Further reduced IgE reactivity may be defined as this polypeptide (h) according to the invention having a greater reduction in the measured degree and/or extent of interaction of IgE as compared to the polypeptide in (a), (b), (c), (d), (e), (f) and/or (g), according to the invention. The method of measurement of IgE reactivity is obvious to a skilled person in the art, in particular using an ELISA IgE immunoassay and/or any other appropriate quantitative methods in the art.

Homology may be defined as polypeptides that are homologous. In particular homologous polypeptides may be defined as being “substantially identical” to the polypeptide according to the invention. In particular, the polypeptide may have 60%, 65%, 70%, 75%, 80%, 85%, 90%, 91%, 92%, 93%, 94%, 95%, 96%, 97%, 98%, 99% or 100% amino acid sequence homology, in particular percent identity, to the polypeptide according to the invention. More in particular, homologous polypeptides retain the allergenic activity and are found in or isolated from a different species of organisms. It is well known to one skilled in the art that conserved amino acids are usually identical across species and organisms and if not, are very similar in their chemical properties.

The Group 5 allergen may be selected from the group consisting of Blo t 5, Der p 5, Der f 5, Der m 5. In particular, the Group 5 allergen is Blo t 5 allergen. The Group 5 allergen may be defined as including any of the protein isoforms of the allergen, and may be further defined as including naturally-occurring and/or recombinant allergens.

In particular, the polypeptide according to the invention is a polypeptide comprising the amino acid sequence of SEQ ID NO:33. The polypeptide according to the invention may also be a polypeptide comprising at least one of the amino acid substitutions corresponding to Leu57, Arg58, Arg74, Glu75, Glu79, Lys92, Lys94, Glu98, Gln102, Asp105, Lys106 of SEQ ID NO:33. It will be obvious to a skilled person in the art, how to modify the wild type Blo t 5 allergen to produce at least one of the amino acid substitutions which correspond to at least one of the amino acid substitutions present in SEQ ID NO:33.

The polypeptide according to the invention may be a Group 5 allergen is from mite. In particular, the mite is house dust and/or storage mite. The Group 5 allergen may be from at least one organism selected from the group consisting of the Blomia genus, the Dermatophagoides genus, the Euoglyphus genus, the Glycyphagus genus, the Lepidoglyphus genus, the Acarus genus and/or the Tyrophagus genus. In particular, the Group 5 allergen is from Blomia tropicalis.

The Blomia genus may be defined in particular as including any one or all of the species Blomia tropicalis, Blomia kulagini, Blomia tjibodas, and/or Blomia thori.

The Dermatophagoides genus may be defined in particular as including any one or all of the species Dermatophagoides pteronyssinus, Dermatophagoides farinae and/or Dermatophagoides microceras.

The Euoglyphus genus may be defined in particular as including the species Euoglyphus maynei. The Glycyphagus genus may be defined in particular as including the species Glycyphagus domesticus.

The Lepidoglyphus genus may be defined in particular as including the species Lepidoglyphus destructor.

The Acarus genus may be defined in particular as including any one or all of the species Acarus siro and/or Acarus farris.

The Tyrophagus genus may be defined in particular as including any one or all of the species Tyrophagus putrescentiae and/or Tyrophagus longior.

In particular, the isolated polypeptide, derivative, isoform and/or fragment thereof, according to the invention may be at least one polypeptide, derivative, isoform and/or fragment thereof comprising at least one amino acid sequence selected from the group consisting of SEQ ID NO:17, SEQ ID NO:19, SEQ ID NO:21, SEQ ID NO:23, SEQ ID NO:25, SEQ ID NO:27, SEQ ID NO:29, SEQ ID NO:31, and/or SEQ ID NO:33.

There is also provided an isolated nucleic acid molecule encoding at least one polypeptide according to the invention. In particular, the isolated nucleic acid molecule is selected from the group consisting of SEQ ID NO:16, SEQ ID NO:18, SEQ ID NO:20, SEQ ID NO:22, SEQ ID NO:24, SEQ ID NO:26, SEQ ID NO:28, SEQ ID NO:30, and/or SEQ ID NO:32. An isolated nucleic acid according to the invention may be defined as a single or double stranded oligonucleotide or polypeptide, or can also be single or double stranded genomic DNA, cDNA, RNA, mRNA.

There is also provided a vector comprising at least one nucleic acid molecule according to the invention. A vector may be defined in particular as an expression vector. Suitable expression vectors may include plasmids and viral vectors such as herpes viruses, retroviruses, vaccinia viruses, attenuated vaccinia viruses, canary pox viruses, adenoviruses and adeno-associated viruses. The vector may further comprise a regulatory nucleotide sequence linked to the nucleic acid molecule according to the invention. The regulatory nucleotide sequence may be a prokaryotic or eukaryotic promoter.

There is also provided a host cell comprising at least one nucleic acid molecule according to the invention and/or at least one vector according to the invention. The host cell may be a prokaryotic and/or eukaryotic cell. The host cell may be defined in particular as a host cell from a bacterium, yeast, mould or animal.

There is also provided a method for producing any polypeptide according to the invention. In particular, the method comprises the step of culturing the host cell according to the invention in a medium and isolating the polypeptide from the culture. The step of culturing the host cell is well known to a skilled person in the art. The step of isolating the polypeptide from the culture is also well known to a skilled person in the art.

Methods for the preparation of a vector comprising the nucleic acid molecule according to the invention, preferably linked to a promoter, and cultivation of the host cell comprising the vector according to the invention can be carried out according to standard techniques. For example, as indicated or described in Sambrook and Russel, Molecular Cloning, Cold Spring Harbour, 2002.

There is also provided an isolated oligonucleotide comprising at least one of the nucleic acid sequences selected from the group consisting of SEQ ID NO: 1 to SEQ ID NO: 15.

There is also provided a pharmaceutical composition comprising an effective amount of at least one polypeptide according to the invention. The pharmaceutical composition may further comprise at least one pharmaceutically acceptable carrier, diluent, adjuvant, excipients, or a combination thereof. Examples of suitable excipients are water, saline, dextrose, glycerol, ethanol and the like as well as combinations thereof. Such a pharmaceutical composition may consist of the active ingredient alone, in a form suitable for administration to a subject, or alternatively the pharmaceutical composition may comprise the active ingredient and one or more pharmaceutically acceptable carrier, excipient and/or diluent.

The pharmaceutical composition may be for local, subcutaneous, intravenal, topical to a tissue locus, parenteral and/or oral administration. for oral administration may suitably be formulated with excipients normally employed for such formulations, e.g. pharmaceutical grades of mannitol, lactose, starch, magnesium stearate, sodium saccharine, cellulose, magnesium carbonate and the like. The pharmaceutical composition may be administered through subcutaneous and/or intramuscular injection. For oral administration, the pharmaceutical composition may be formulated as solutions, suspensions, emulsions, tablets, pills, capsules, sustained release formulations, aerosols, powders, or granulates.

There is also provided a vaccine against allergic reactions elicited by a naturally occurring allergen in patients suffering from allergy, comprising at least one polypeptide according to the invention. The vaccine according to the invention may be administered in a way that is compatible with the dosage formulation and in such amount as will be therapeutically effective and immunogenic. The quantity of active component contained within the vaccine depends on the subject to be treated, i.e. the capability of the subject's immune system to respond to the treatment, the route of administration and the age and weight of the subject. The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery. Suitable dosage ranges may vary within the range from 0.0001 μg to 1000 μg.

The vaccine may further comprise at least one adjuvant. The adjuvant may be any suitable therapeutically effective adjuvant. Preparation of vaccines with selection of suitable adjuvants is generally well known in the art. In particular, the vaccine may further comprise adjuvants such as aluminum hydroxide and phosphate (alum) or calcium phosphate as a 0.05 to 0.1 percent solution in phosphate buffered saline, synthetic polymers of sugars or polylactid glycolid (PLG) used as 0.25 percent solution. Mixture with bacterial cells such as C. parvum, endotoxins or lipopolysaccharide components of gram-negative bacteria, emulsion in physiologically acceptable oil vehicles such as mannide monoaleate (Aracel A) or emulsion with 20 percent solution of a perfluorocarbon (e.g. Fluosol-DA) used as a block substitute may also be employed. Oil emulsions, such as MF-59 may also be used. Other adjuvants such as Freund's complete and incomplete adjuvants as well as saponins, such as QuilA, Qs-21 and ISCOM, and RIBI may also be used. The vaccine may additionally contain other substances such as wetting agents, emulsifying agents, buffering agents or adjuvants enhancing the effectiveness of the vaccine.

In particular, multiple administrations of the vaccine may be necessary to ensure an effect. It is known in the art that frequently the vaccine is administered as an initial administration followed by subsequent inoculations or other administrations. The number of vaccinations may be in the range of from 1 to 50, usually not exceeding 35 vaccinations. Vaccination may normally be performed from biweekly to bimonthly for a period of 3 months to 5 years.

There is also provided a process for preparing a pharmaceutical composition according to the invention comprising mixing at least one polypeptide according to the invention with at least one pharmaceutically acceptable substance and/or excipients.

There is also provided at least one polypeptide according to the invention for use in medicine.

There is also provided at least one polypeptide for the preparation of a medicament for the prevention and/or treatment of allergy. In particular, the allergy may be Type I allergy. Type I allergy may be characterised by excessive activation of mast cells and basophils by IgE resulting in a systemic inflammatory response that may result in symptoms as benign as a runny nose, to life-threatening anaphylactic shock and death. In particular, the preparation may be suitable for providing protection against allergic responses during the period of the year where symptoms occur (prophylaxis). Usually, the treatment may have to be repeated every year to maintain the protective effect. Preparations formulated for nasal, oral and sublingual application may be particularly suited for this purpose.

There is also provided the use of at least one polypeptide according to the invention for the preparation of a medicament for specific allergen immunotherapy (SIT).

There is also provided an isolated antibody, wherein the antibody specifically binds to at least one polypeptide according to the invention. An antibody is any immunoglobulin, including antibodies and fragments thereof, that binds to a specific epitope. The antibody according to the invention may be prepared against the polypeptide according to the invention, a derivative and/or a fragment thereof. Such antibodies include, but are not limited to polyclonal, monoclonal, chimeric, humanised, single chain, Fab, Fab′, F(ab)′ fragments and/or F(v) portions of the whole antibody.

Various procedures known in the art may be used for the production of polyclonal antibodies to the polypeptide of the invention, or immunogenic fragment thereof. For the production of antibody, various host animals can be immunised by injecting the polypeptide or an immunogenic fragment thereof, including but not limited to rabbits, mice, rats, sheep, goats, etc. The polypeptide of the invention, a derivative and/or fragment thereof may be conjugated to an immunogenic carrier, e.g., bovine serum albumin (BSA) or keyhole limpet hemocyanin (KLH). The polypeptide of the invention or immunogenic fragment may be further combined with any adjuvant known in the art (for example, Hood et al., in Immunology, p. 384, Second Ed., Benjamin/Cummings, Menlo Park, Calif., 1984, herein incorporated by reference).

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffision assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, and the like. Antibody binding may be detected by detecting a label on the primary antibody, and/or by detecting the binding of a secondary antibody or reagent to the primary antibody. Alternatively, the secondary antibody may be labelled. Many means are known in the art for detecting the binding in an immunoassay.

There is also provided a kit comprising at least one polypeptide according to the invention. There is also provided a kit comprising at least one pharmaceutical composition and/or a vaccine according to the invention. The kit may be an immunotherapeutic kit.

There is also provided a diagnostic assay for assessing relevance, safety or outcome of therapy of a subject using at least one polypeptide according to the invention and/or at least one antibody according to the invention, wherein at least one IgE containing sample of the subject is mixed with the polypeptide and/or antibody and assessed for the level of IgE reactivity. A pharmaceutical composition according to the invention intended to be administered therapeutically may also be used for an in vivo or in vitro diagnostic assay to monitor the relevance, safety and/or outcome of a treatment with such mutants or compositions. Diagnostic samples to be applied include body samples, such as sera. The assessing of the level of reactivity between the IgE in the sample and the mutant may be carried out using any known immunoassay. The use of an immunoassay is well known to a skilled person in the art.

The polypeptide according to the invention may have diagnostic possibilities and advantages. Prior art allergy vaccines may be based on extracts of the naturally occurring allergen source, and thus represent a wide variety of isoforms. The allergic subject may initially have been sensitised and may have IgE to one or some of the isoforms present. Some of the isoforms may be relevant with respect to the allergic reactions of the allergic subject due to homology and subsequent cross-reactivity with the isoform to which the subject is allergic, whereas other isoforms may be irrelevant if they do not harbour any of the IgE binding epitopes to which the allergic subject has specific IgE. Due to this heterogeneity of the specificities of the IgE population, some isoforms may therefore be safe to administer, i.e. they do not result in an allergic response via IgE, whereas other isoforms may be harmful causing undesirable side effects.

The polypeptide provided in the invention and the compositions of the invention is comprised of a hypoallergenic isoforms that do not result in an allergic response via IgE as above.

Having now generally described the invention, the same will be more readily understood through reference to the following examples; which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

In particular, the present inventors prepared hypoallergenic Blo t 5 derivatives for safer and effective allergen specific immunotherapy of mite allergy.

Multiple mutations were introduced by recombinant polymerase chain reaction to modify Blo t 5, a major mite allergen from Blomia tropicalis. The immunoglobin E (IgE) binding capacity of the mutated Blo t 5 was assessed in vitro and in vivo using human IgE ELISA, Ig E ELISA inhibition, histamine release, and skin prick tests.

To examine the T-cell cytokine profiles upon stimulation with native and mutant Blo t 5, human T-cell studies were performed with peripheral blood mononuclear cells (PBMC) taken from allergic subjects who had positive skin prick tests with Blo t 5, and non-allergic subjects. Secondary structure of the mutant protein was examined by circular dichroism (CD) analysis.

A panel of nine Blo t 5 mutants were generated and characterized.

Among these mutants, mutant M9 (SEQ ID NO: 33) showed the most significant reduction in IgE reactivity when it was tested with a panel of patients' sera by human IgE ELISA. The reduction in allergenicity of M9 was further confirmed by in vivo skin prick tests and in vitro histamine release tests.

In addition, M9 mutant allergen induced and enhanced the production of IL-10 and IFN-gamma cytokines, but not IL-4, IL-5 and IL-13 cytokines, by T cells from mite allergic patients. This cytokine profiling data demonstrated that mutant M9 is immunogenic by retaining its ability to stimulate T cells in an antigen specific way despite its reduced allergenicity. More importantly, the data also revealed that M9 exhibited modified immunogenic properties (i.e. enhanced production of IL-10 and IFN-gamma) that have beneficial immunomodulatory effects for allergy immunotherapy. In conclusion, M9 is potentially a good candidate for the development of a safer and more effective immunotherapeutic reagent.

Accordingly, the inventors prepared hypoallergenic derivatives of recombinant allergens, which exhibited reduced IgE reactivity but retained the capacity to stimulate T-cells to induce beneficial responses that are favourable for immunotherapy.

The present inventors demonstrated that a considerable loss of IgE binding was achieved with 11 point mutations (M9) of Blo t 5. The lowered IgE reactivity of M9 also resulted in a reduction in allergenic properties, which was shown in the basophil histamine release experiment conducted and the skin prick testing in B. tropicalis allergic subjects. Elevated levels of IL-10 and IFN-gamma in the T cells stimulated with M9 revealed that in this mutant, the immunogenic properties of Blo t 5 have been altered favourably. Accordingly, M9 is a suitable candidate for SIT, because it enhances the efficacy of immunotherapy.

### Sera

Sera was obtained from 30 children (aged 4 to 15 years old) seen at the National University Hospital's Children's Specialist Clinic, with clinically diagnosed allergic disorders (asthma, rhinitis and or eczema). All had positive skin prick test (SPT) reactions to B. tropicalis and D. pteronyssinus. Serum from a non-sensitized subject was used as control.

### Subjects

Thirty-eight skin-tested B. tropicalis-positive subjects (28 females and 10 males, aged 10 to 49 years old) have been recruited for skin prick test of Blo t 5 and M9. Three non-allergic subjects were included as negative control. Peripheral blood mononuclear cells of 5 Blo t 5 and 1 non-allergic subjects were used for T cell cytokine in this study.

### Construction of Mutated Blo T 5 in Expression Vector

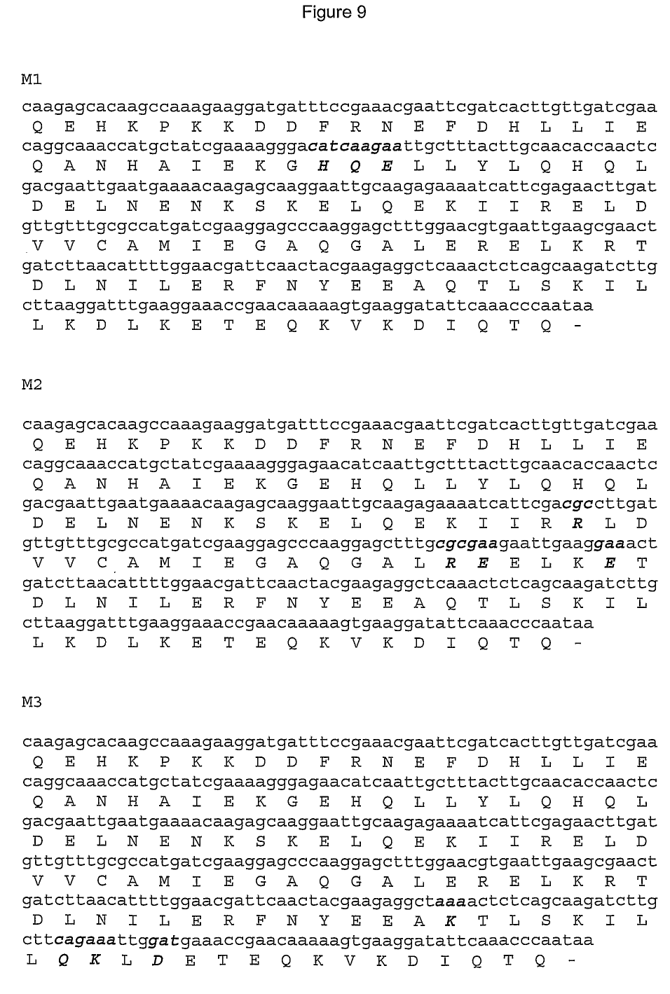

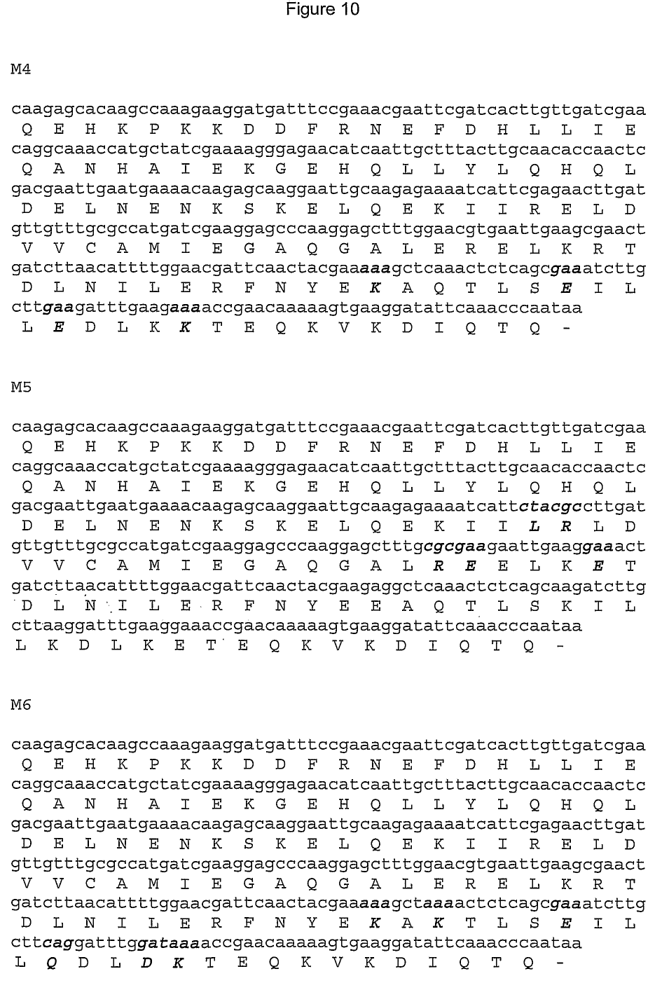

A cDNA encoding for the Blo t 5 protein was cloned into the Bam HI and Xho I sites of pGEX-4T1 vector. This DNA template was used to generate a panel of mutants by recombinant-PCR method. Secondary structure prediction by PHD prediction program showed that Blo t 5 is an α-helical protein. Internal amino acid substitutions were engineered in order to disrupt the structure of Blo t 5, and hence reduced or abolished IgE binding. Oligonucleotide primers used for Blo t 5 mutants construction are listed in Table 1. Codons for amino acids changed are underlined. FIGS. 9 and 10 show the nucleotide and amino acid sequences of the mutants M1 to M6. Primers 1 and 2 were used to produce M1; primers 3 and 4 for M2; primers 5 and 6 for M3, and M4 was produced by primers 7 and 8. The cDNA fragments produced were cloned into the Bam HI and Xho I sites of pGEX-4T1 vector and transformed into E. coli DH5α.

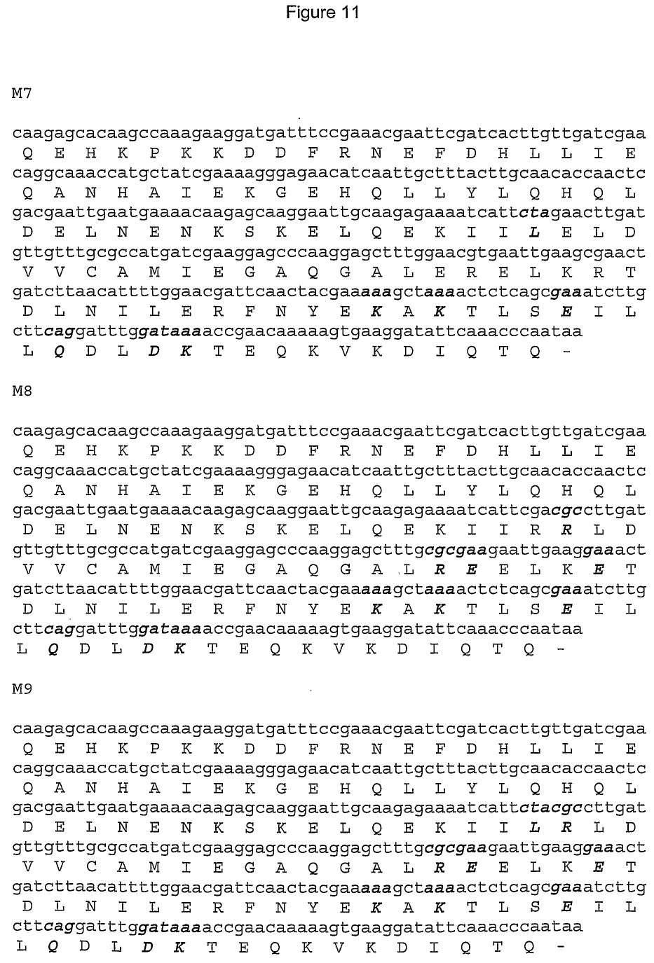

The same strategy was used to construct and clone the combinatory mutants (FIGS. 10 and 11: M5, M6, M7, M8, M9). Primers 3, 9 to 13 were used to construct these mutants. The sequence of these clones were then checked and confirmed. Recombinant proteins were expressed as glutathione-S-transferase fusion proteins. Table 2 showed the location of amino acids substitution in the Blo t 5 mutants constructed. FIGS. 10 and 11 show the nucleotide and amino acid sequences of the mutants M4 to M9.

TABLE 1
List of primer sequences used

| SEQ ID: | Primer | |
|---|---|---|
| NO | designation | Sequences |
| 1 | A-F | 5′ GCTATCGAAAAGGGACATCAAGAATTGCTTTACTTGCAA 3′ |
| 2 | A-R | 5′ TTGCAAGTAAAGCAATTCTTGATGTCCCTTTTCGATAGC 3′ |
| 3 | B-F | 5′ ATGATCGAAGGAGCCCAAGGAGCTTTGCGCGAAGAATTGAAGGAAA |
| | | CTGATCTTAACATT 3′ |
| 4 | B-R | 5′ TCCTTGGGCTCCTTCGATCATGGCGCAAACAACATCAAGGCGTCGAA |
| | | TGATTTTCTC 3′ |
| 5 | C1-F | 5′ ACTCTCAGCAAGATCTTGCTTCAGAAATTGGATGAAACCGAACAA 3′ |
| 6 | C1-R | 5′ AAGCAAGATCTTGCTGAGAGTTTTAGCCTCTTCGTA 3′ |
| 7 | C2-F | 5′ AAAGCTCAAACTCTCAGCGAAATCTTGCTTGAAGATTTGAAGAAA |
| | | ACCGAACAAAAAGTG 3′ |
| 8 | C2-R | 5′ TTCGCTGAGAGTTTGAGCTTTTTCGTAGTTGAA 3′ |
| 9 | L/B-R | 5′ TCCTTGGGCTCCTTCGATCATGGCGCAAACAACATCAAGGCGTAG |
| | | AATGATTTTCTC 3′ |
| 10 | C1C2-F | 5′ CTCAGCGAAATCTTGCTTCAGGATTTGGATAAAACCGAACAAAAA |
| | | GTG 3′ |
| 11 | C1C2-R | 5′ CTGAAGCAAGATTTCGCTGAGAGTTTTAGCTTTTTCGTAGTTGAA 3′ |
| 12 | G4Fa | 5′ GTGTTTCGAGAATTGCATATAGT 3′ |
| 13 | G4Ra | 5′ ACCTGACGTCTAAGAAACCATT 3′ |
| 14 | pGEX-F | 5′ GGG CTG GCA AGC CAC GTT TGG TG 3′ |
| 15 | pGEX-R | 5′ CCG GGA GCT GCA TGT GTC AGA GG 3′ |

TABLE 2
List of amino acid substitutions for the constructs

| SEQ ID NO: | | | | |
|---|---|---|---|---|
| Nucleic acid | Amino acid | Code | Constructs | Amino acid change |
| 16 | 17 | M1 | geA | His30, Gln31, Glu32 |
| 18 | 19 | M2 | geB | Arg58, Arg74, Glu75, Glu79 |
| 20 | 21 | M3 | geC1 | Lys94, Gln102, Lys103, Asp105 |
| 22 | 23 | M4 | geC2 | Lys92, Glu98, Glu102, Lys106 |
| 24 | 25 | M5 | CM1 (L/B) | Leu57, Arg58, Arg74, Glu75, Glu79 |
| 26 | 27 | M6 | CM2 (C1C2) | Lys92, Lys94, Glu98, Gln102, Asp105, |
| | | | | Lys106 |
| 28 | 29 | M7 | CM3 (LC1C2) | Leu57, Lys92, Lys94, Glu98, Gln102, |
| | | | | Asp105, Lys106 |
| 30 | 31 | M8 | CM4 (BC1C2) | Arg58, Arg74, Glu75, Glu79, Lys92, |
| | | | | Lys94, Glu98, Gln102, Asp105, Lys106 |
| 32 | 33 | M9 | (CM5) LBC1C2 | Leu57, Arg58, Arg74, Glu75, Glu79, |
| | | | | Lys92, Lys94, Glu98, Gln102, Asp105, |
| | | | | Lys106 |

### Expression of Recombinant Proteins

Expression of Blo t 5 mutants as fusion protein with glutathione-S-transferase (GST) was produced by culturing transformed E. coli in LB media until OD600=0.6. One millimolar of IPTG was added for induction. The fusion proteins were affinity-purified using glutathione agarose (Sigma) and followed by elution with 10 mM reduced glutathione. GST was then cleaved from the fusion proteins with thrombin (1 unit for 4 O.D.) at 4 C, overnight. GST was removed by glutathione agarose. The collected fractions were dialysed against PBS and kept at −20° C.

### Circular Dichroism (CD) Analysis

CD was performed at 25° C. using a CD spectrometer model 202 (AVIV instrument Inc., Lakewood, N.J., USA) equipped with constant with constant liquid nitrogen flush. Proteins were measured in 10 mM sodium phosphate pH 7.0 in 0.1 cm path length cuvette over the far-UV range of 260 to 190 nm. The results from the average of three scans were presented as mean residual ellipticities, ⊖.

### Elisa IgE Immunoassay, Dose-Dependent Inhibition Assay and Peptides Absorption Assay

ELISA was performed by coating each well with 250 ng of fusion proteins in 0.1 M sodium bicarbonate buffer, pH 8.3, overnight at 4° C. The wells were washed 3 times with PBST-T (0.05% of Tween 20 in PBS) before blocking with 1% bovine serum albumin (Sigma) in PBST for 1 h at room temperature, followed by overnight incubation at 4° C. with diluted (5 to 30×) human sera in blocking solution. Specific IgE was detected by incubating 0.25 μg/mL biotinylated mouse anti-human IgE (Southern Biotechnology Associates Inc, Birmingham, Ala., USA) for 1 hour at room temperature after 3 times of washing, and followed by incubating 1:2000 diluted ExtrAvidin-alkaline phosphatase (Sigma) at room temperature for an additional 1 hour. Signal was developed by addition of p-nitro-phenylphosphate (Sigma). The optical density was read at 405 nm.

ELISA inhibition assays were performed by prior overnight absorption of positive sera in 100 μg/ml of Blo t 5, M9, or GST. Human sera used were 5 to 30 times dilution.

### Skin Prick Tests

Skin prick tests were conducted according to the protocol described previously (Korematsu et al. 2000). Briefly, the skin of the volar aspect of the forearm was pricked with a disposable lancet with the presence of an allergen droplet. The prick was regarded positive when a wheal diameter was 3 mm greater than the negative control. Glycerol-buffer (25 μg/mL), and 1 mg/mL of histamine were used as negative and positive controls, respectively. All the purified allergens were used at 25 μg/mL in this study.

### T Cell Culture

Twenty-five millilitres of blood was diluted with an equal volume of RPMI (Gibco, New York, N.Y., USA) and peripheral blood mononuclear cells (PBMC) were isolated on a density gradient Ficoll-Paque PLUS (Pharmacia). The cells were seeded at 2×10⁵ cells per well in a 96-well round bottom trays, in triplicate, in a final volume of 200 μL AIM-V culture medium (Gibco) at 37° C. in 5% CO₂. Wild-type Blo t 5 and M9 proteins were added at 10 μg/mL. Culture supernatants were harvested on day 3 and 6, and kept at −80° C.

### T Cell Cytokine Assays

Cytokines were measured in thawed supernatants by particle-based immunoassay. IL-4, IL-5, IL-10, and IFN-gamma were measured using BD Cytometric Bead Array system from BD Biosciences (San Diego, Calif., USA). BD Human Th2 Cytokine CBA Kit (BD Biosciences) was used in this study. The assay was prepared according to manufacturer manual. Briefly, reconstituted human Th2 cytokines standards in the assay diluent provided and followed by serial dilution. Ten micro-litres of each human cytokine capture bead suspension were mixed and 50 μl of the mixed beads were transferred to assay tubes. Fifty micro-litres of PE detection reagent was added into the assay tubes and followed by adding 50 μl of the diluted standards and culture supernatants. The mixtures were then incubated at room temperature for 3 hours. The samples were then washed and centrifuge. The supernatants were discarded. Three-hundred micro-litres of wash buffer were added before analyzing the samples on a flow cytometer (FACScalibur). The cytokines detection sensitivity of cytokines in the kit was 20 pg/ml.

### Histamine Release Assay

Blo t 5 and M9 proteins were diluted in a histamine release reaction buffer to concentration ranging from 0.001 pg/ml to 10 μg/ml. Histamine release was performed with histamine release in heparinized whole blood kit (IBL, Hamburg, Germany), according to the manufacturer's recommendations. Briefly, 200 μl heparinized whole blood samples were incubated at 37° C. for 1 hour with different concentrations of the allergen. Release of histamine will occur upon stimulation of basophilic granulocytes depending on their sensitivity to the allergen. The released histamine in the supernatant was subsequently determined using a specific plasma immunoassay, the histamine ELISA (IBL), according to the manufacturer's instructions.

### Destruction of IgE Epitopes in M9

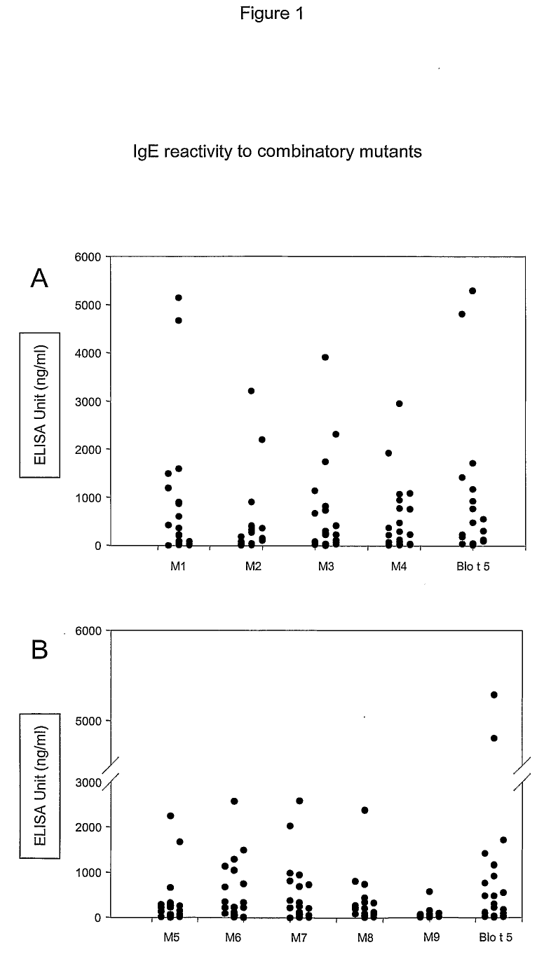

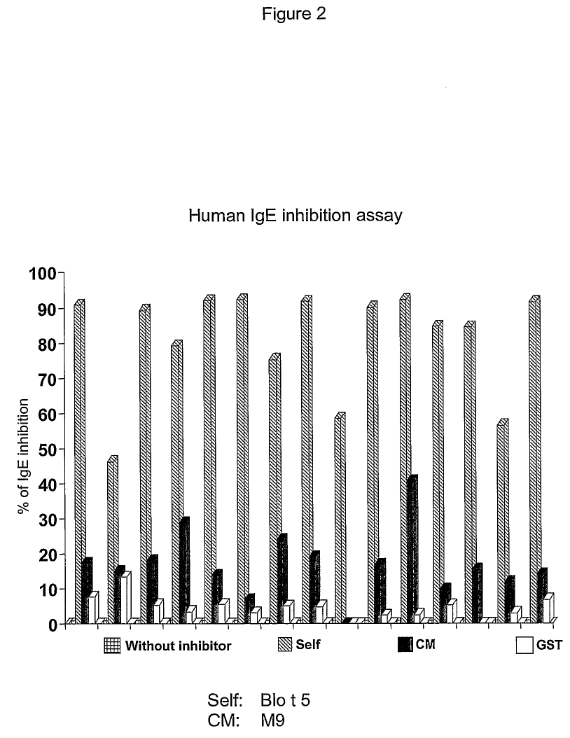

No reduction in IgE-binding was observed in the three point mutations in M1 (Table 2). Slight but not significant reduction in IgE-binding to M2, M3 and M3 (FIG. 1, graph A) was observed. There is no significant IgE-binding reduction in all of the combinatory mutants, except for M9, where p<0.02 (FIG. 1, graph B). Human IgE inhibition assay was performed by using 15 Blo t 5-positive subjects. Reduction in percentage of IgE inhibition for M9 showed that this combinatory mutant has no ability to inhibit the IgE binding to Bt5A. This observation further confirmed that the IgE epitopes of M9 has been disrupted (FIG. 2), i.e. no IgE-binding. Eighty percent (12 out of 15) of the subjects tested showed less than 20% inhibition of Blo t 5 by M9, with one subject having no inhibition at all.

α-Helical Structure of M9 has been Destroyed

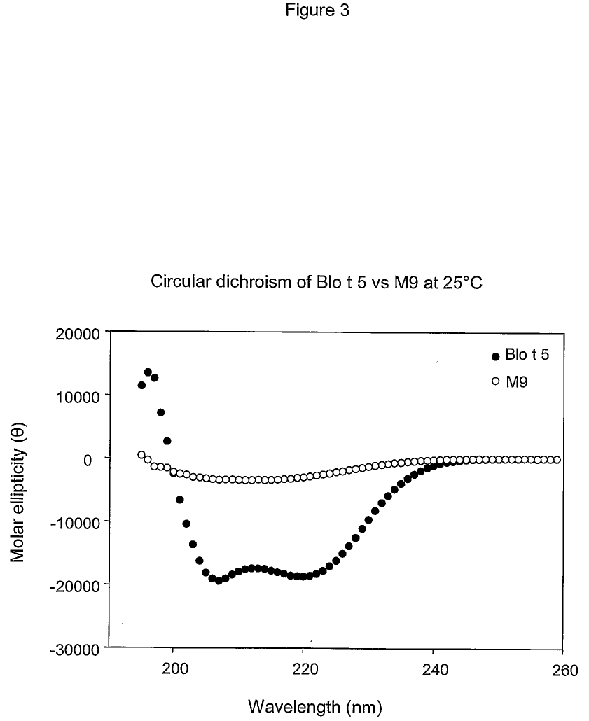

The far-UV spectrum of Blo t 5 (Bt5A) was characterized by minima at 207 nm and 220 nm (FIG. 3). This shape is a typical well-structured protein with a considerable amount of α-helices. The circular dichroism results are in good agreement with secondary structure prediction using the PHD package. The spectra of the mutant, M9 showed that the α-helical structure of M9 has been destroyed.

### Reduction in Allergenicity of M9

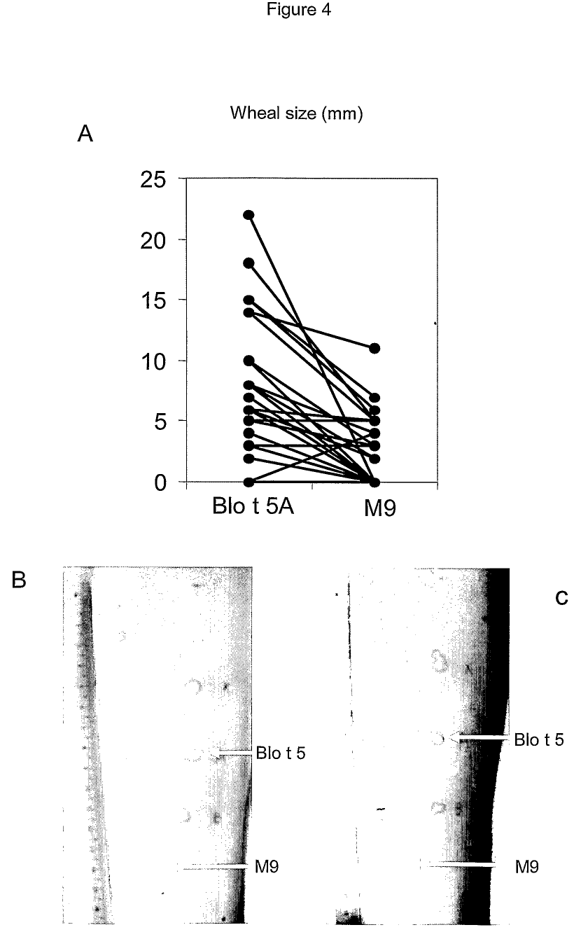

The capacity of Blo t 5 and M9 to elicit immediate skin reactions was evaluated in 38 B. tropicalis skin test positive subjects. FIG. 4 showed that the results of skin prick test, where 37 out of 38 subjects reduction in skin reactivity with M9, and only one subject tested has a slight increase in skin reactivity against M9. Twelve of the subjects tested showed no skin reactivity to M9. FIG. 4 graph A shows the wheal size of subjects tested, and FIG. 4 photos (B) shows the difference in skin reactivity to Blo t 5 and M9, indicated by arrows (upper arrow—Blo t 5; lower arrow—M9).

### Reduced Ability of M9 to Induce Histamine Release

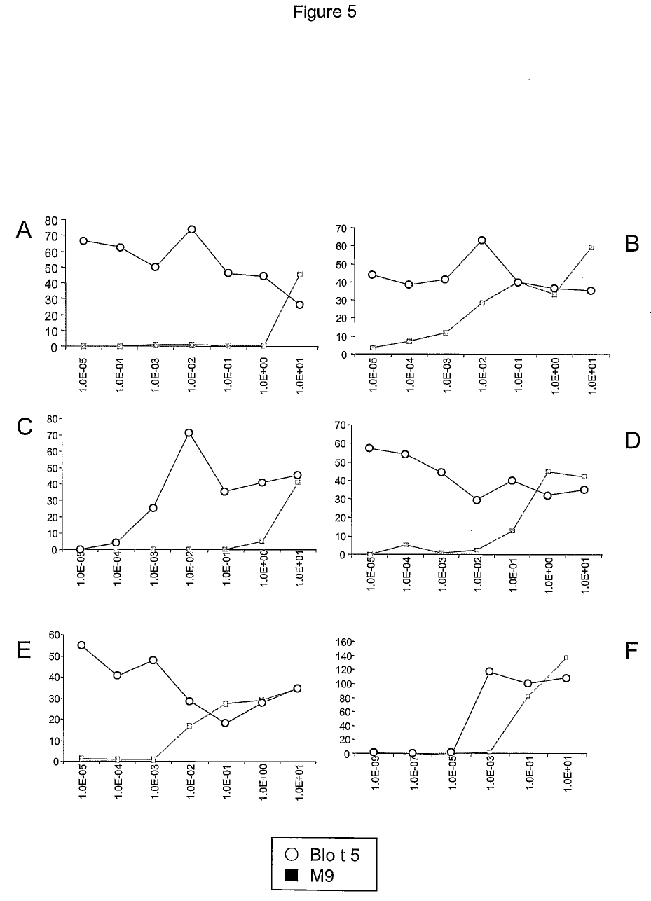

To evaluate the capacity of Blo t 5 and M9 to induce histamine release, whole blood from B. tropicalis allergic subjects were challenged with various concentrations (0.001 pg/mL to 10 μg/mL) of Blo t 5 and M9. A typical dose-dependent histamine release (FIG. 5) from basophils was observed in all allergic subjects. The results of histamine release study showed that there are at least 100 fold or more reduction of allergenic activity of M9 to induce histamine release in 7 of the subjects tested (FIG. 5), than Blo t 5.

Basophils of one of the subjects required as high as 1 μg/ml of M9 in order to induce the release of histamine.

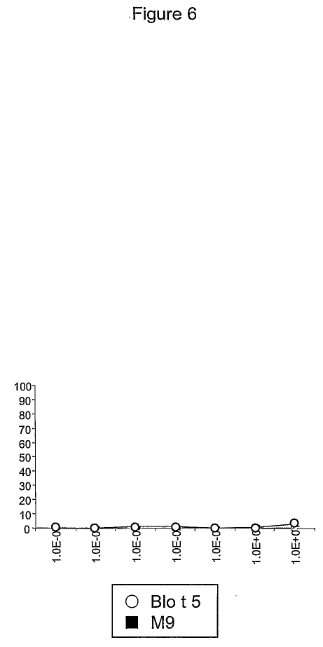

No histamine release in response to native and recombinant Blo t 5 was triggered when basophils from a non-allergic subject was used (FIG. 6).

M9 Induces Higher IFN-Gamma and IL-10 in the Peripheral Blood Mononuclear Cells of Blo T 5 Positive Subjects

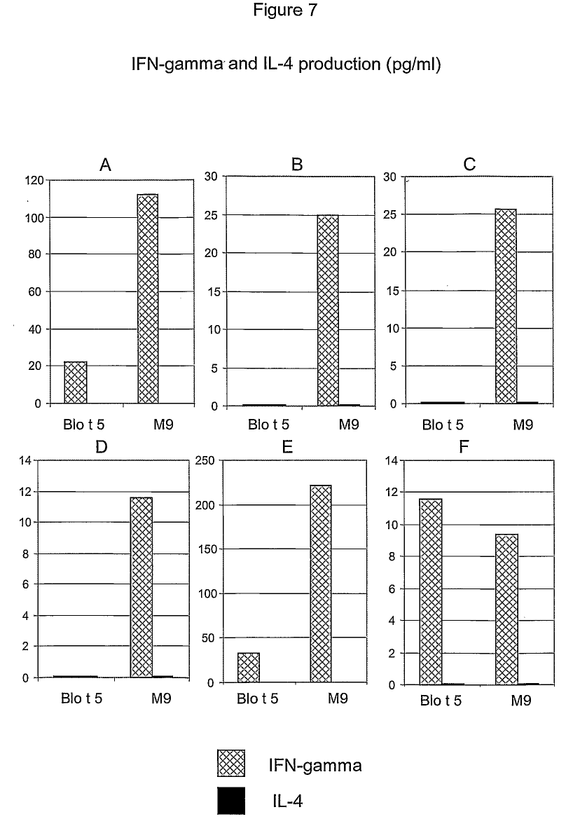

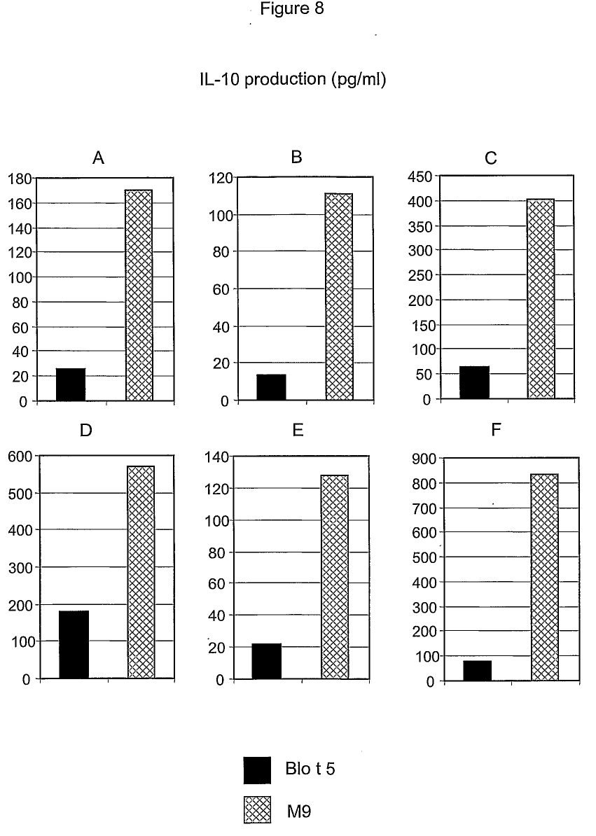

T-cell cytokines analysis showed that both allergic and non-allergic subjects that had been stimulated with Blo t 5 and M9 did not produce enough IL-4 even to be detectable. It was found that the mutant protein M9 is able to stimulate T cells to produce far more IFN-gamma than Blo t 5 in allergic subjects (FIG. 7) In contrast, there is no difference in the level of IFN-gamma produced by non-allergic T cells after stimulating with both Blo t 5 and M9. Results also showed that peripheral blood mononuclear cells (PBMC) of Blo t 5 positive subjects stimulated with M9 is able to produce higher IL-10 in all subjects (allergic and non-allergic) than Blo t 5 (FIG. 8).

### REFERENCES

Aalberse R C, van der Gaag R, van Leeuwen J. Serologic aspects of IgG4 antibodies. I. Prolonged immunization results in an IgG4-restricted response. J. Immunol. February; 130(2):722-6 (1983)
Abramson M J, Puy R M, Weiner J M. Allergen immunotherapy for asthma. Cochrane Database Syst Rev. 2000(2):CD001186 (2000)
Akdis C A, Blaser K. IL-10-induced anergy in peripheral T cell and reactivation by microenvironmental cytokines: two key steps in specific immunotherapy. FASEB J. April; 13(6):603-9 (1999)
Akdis C A, Blesken T, Akdis M, Wuthrich B, Blaser K. Role of interleukin 10 in specific immunotherapy. J Clin Invest. July 1; 102(1):98-106 (1998)
Arruda L K, Fernandez-Caldas E, Naspitz C K, Montealegre F, Vailes L D, Chapman M D. Identification of Blomia tropicalis allergen Blo t 5 by cDNA cloning. Int Arch Allergy Immunol; 107:456-7 (1995)
Bellinghausen I, Metz G, Enk A H, Christmann S, Knop J, Saloga J. Insect venom immunotherapy induces interleukin-10 production and a Th2-to-Th1 shift, and changes surface marker expression in venom-allergic subjects. Eur J. Immunol. May; 27(5):1131-9 (1997)
Bousquet J, Lockey R F, Malling H-J, edsWHO Position Paper Allergen Immunotherapy: Therapeutic Vaccines for Allergic Diseases. Allergy; 53 (Suppl. 44):1 42 (1998)
Bousquet J., R Lockey, and H J Malling. Allergen immunotherapy: therapeutic vaccines for allergic diseases: a WHO position paper. J. Allergy Clin Immunol. 102:558 (1998)
Breiteneder H, Ferreira F, Hoffmann-Sommergruber K, Ebner C, Breitenbach M, Rumpold H, Kraft D, Scheiner O. Four recombinant isoforms of Cor a I, the major allergen of hazel pollen, show different IgE-binding properties. Eur J. Biochem. March 1; 212(2):355-62 (1993)
Creticos P S, Adkinson N F Jr, Kagey-Sobotka A, Proud D, Meier H L, Naclerio R M, et al. Nasal challenge with ragweed pollen in hay fever patients. Effect of immunotherapy. J Clin Invest; 76:2247-53 (1985)
de Silva H D, Gardner L M, Drew A C, Beezhold D H, Rolland J M, O'Hehir R E. The hevein domain of the major latex-glove allergen Hev b 6.01 contains dominant T cell reactive sites. Clin Exp Allergy. April; 34(4):611-8 (2004)
de Waal Malefyt R, Abrams J, Bennett B, Figdor C G, de Vries J E. Interleukin 10(IL-10) inhibits cytokine synthesis by human monocytes: an autoregulatory role of IL-10 produced by monocytes. J Exp Med. November 1; 174(5):1209-20 (1991)
Del Prete G, De Carli M, Almerigogna F, Giudizi M G, Biagiotti R, Romagnani S. Human IL-10 is produced by both type 1 helper (Th1) and type 2 helper (Th2) T cell clones and inhibits their antigen-specific proliferation and cytokine production. J. Immunol. January 15; 150(2):353-60 (1993)
Devey M E, Wilson D V, Wheeler A W. The IgG subclasses of antibodies to grass pollen allergens produced in hay fever patients during hyposensitization. Clin Allergy. May; 6(3):227-36 (1976)
Durham S R, S M Walker, E M Varga, M R Jacobson, F O'Brien, W Noble, S J Till, Q A Hamid and K T Nouri-Aria. Long-term clinical efficacy of grass-pollen immunotherapy. N Engl J Med 341:468 (1999)
Ferreira F, Hirtenlehner K, Jilek A, Godnik-Cvar J, Breiteneder H, Grimm R, Hoffmann-Sommergruber K, Scheiner O, Kraft D, Breitenbach M, Rheinberger H J, Ebner C. Dissection of immunoglobulin E and T lymphocyte reactivity of isoforms of the major birch pollen allergen Bet v 1: potential use of hypoallergenic isoforms for immunotherapy. Exp Med. February 1; 183(2):599-609 (1996)
Fiorentino D F, Zlotnik A, Vieira P, Mosmann T R, Howard M, Moore K W, O'Garra A. IL-10 acts on the antigen-presenting cell to inhibit cytokine production by Th1 cells. J. Immunol. May 15; 146(10):3444-51 (1991)
Francis J N, Till S J, Durham S R. Induction of IL-10+CD4+ CD25+T cells by grass pollen immunotherapy. J Allergy Clin Immunol. June; 111(6):1255-61 (2003)
Gardner L M, O'Hehir R E, Rolland J M. High dose allergen stimulation of T cells from house dust mite-allergic subjects induces expansion of IFN-gamma+ T Cells, apoptosis of CD4+IL-4+ T cells and T cell anergy. Int Arch Allergy Immunol. January; 133(1):1-13 (2004)
Gardner L M, Thien F C, Douglass J A, Rolland J M, O'Hehir R E. Induction of T ‘regulatory’ cells by standardized house dust mite immunotherapy: an increase in CD4+ CD25+ interleukin-10+ T cells expressing peripheral tissue trafficking markers. Clin Exp Allergy. August; 34(8):1209-19 (2004)
Gehlhar K, Schlaak M, Becker W, Bufe A. Monitoring allergen immunotherapy of pollen-allergic patients: the ratio of allergen-specific IgG4 to IgG1 correlates with clinical outcome. Clin Exp Allergy. April; 29(4):497-506 (1999)
Groux H, O'Garra A, Bigler M, Rouleau M, Antonenko S, de Vries J E, Roncarolo M G. A CD4+ T-cell subset inhibits antigen-specific T-cell responses and prevents colitis. Nature. October 16; 389(6652):737-42 (1997)
Jeannin P, Lecoanet S, Delneste Y, Gauchat J F, Bonnefoy J Y. IgE versus IgG4 production can be differentially regulated by IL-10. J. Immunol. April 1; 160(7):3555-61 (1998)
Jutel M, Akdis M, Budak F, Aebischer-Casaulta C, Wrzyszcz M, Blaser K, Akdis C A. IL-10 and TGF-beta cooperate in the regulatory T cell response to mucosal allergens in normal immunity and specific immunotherapy. Eur J. Immunol. May; 33(5):1205-14 (2003)
Kimura I, Tanizaki Y, Goda Y, Komagoe H, Kitani H. Decrease in reactivity of basophils by immunotherapy with housedust extract. Clin Allergy. January; 15(1):1-7 (1985)
Korematsu S, Tanaka Y, Hosoi S, Koyanagi S, Yokota T, Mikami B, Minato N. C8/119S mutation of major mite allergen Derf-2 leads to degenerate secondary structure and molecular polymerization and induces potent and exclusive Th1 cell differentiation. J. Immunol. September 1; 165(5):2895-902 (2000)
Kuo I C, Cheong N, Trakultivakorn M, Lee B W, Chua K Y. An extensive study of human IgE cross-reactivity of Blo t 5 and Der p 5. J Allergy Clin Immunol. March; 111(3):603-9 (2003)
Kusunoki T, Inoue Y, Korematsu S, Harazaki M, Yokota T, Hosoi S. Comparison of skin prick test with serially diluted wild-type and genetically engineered recombinant Der f2. Ann Allergy Asthma Immunol. March; 84(3):366-8 (2000)
Mailing H. J., Immunotherapy as an effective tool in allergy treatment. Allergy 1998; 53:461 (1998)
Mailing H J, Weeke B. EAACI Immunotherapy position papers. Allergy; 48 (Suppl. 14):9 35 (1993)
Marcotte G V, Braun C M, Norman P S, Nicodemus C F, Kagey-Sobotka A, Lichtenstein L M, Essayan D M. Effects of peptide therapy on ex vivo T-cell responses. J Allergy Clin Immunol. April; 101(4 Pt 1):506-13 (1998)
Miyamoto T., Increased prevalence of pollen allergy in Japan. In Godard P., Bousquet J., and Michel F. B., (Eds.) Advances in Allergology and Clinical Immunology. The Parthenon Publishing Group, Cornforth., pp 434-437. (1992)
Niederberger V, Horak F, Vrtala S, Spitzauer S, Krauth M T, Valent P, Reisinger J, Pelzmann M, Hayek B, Kronqvist M, Gafvelin G, Gronlund H, Purohit A, Suck R, Fiebig H, Cromwell O, Pauli G, van Hage-Hamsten M, Valenta R. Vaccination with genetically engineered allergens prevents progression of allergic disease. Proc Natl Acad Sci USA. October 5; 101 Suppl 2:14677-82 (2004)
Nouri-Aria K T, Wachholz P A, Francis J N, Jacobson M R, Walker S M, Wilcock L K, Staple S Q, Aalberse R C, Till S J, Durham S R. Grass pollen immunotherapy induces mucosal and peripheral IL-10 responses and blocking IgG activity. J Immunol. March 1; 172(5):3252-9 (2004)
Pauli G, Purohit A, Oster J P, De Blay F, Vrtala S, Niederberger V, Kraft D, Valenta R. Comparison of genetically engineered hypoallergenic rBet v 1 derivatives with rBet v 1 wild-type by skin prick and intradermal testing: results obtained in a French population. Clin Exp Allergy. August; 30(8):1076-84 (2000)
Pene J, Desroches A, Paradis L, Lebel B, Farce M, Nicodemus C F, Yssel H, Bousquet J. Immunotherapy with Fel d 1 peptides decreases IL-4 release by peripheral blood T cells of patients allergic to cats. J Allergy Clin Immunol. October; 102(4 Pt 1):571-8 (1998)
Pierkes M, Bellinghausen I, Hultsch T, Metz G, Knop J, Saloga J. Decreased release of histamine and sulfidoleukotrienes by human peripheral blood leukocytes after wasp venom immunotherapy is partially due to induction of IL-10 and IFN-gamma production of T cells. J Allergy Clin Immunol. February; 103(2 Pt 1):326-32 (1999)
Punnonen J, de Waal Malefyt R, van Vlasselaer P, Gauchat J F, de Vries J E. IL-10 and viral IL-10 prevent IL-4-induced IgE synthesis by inhibiting the accessory cell function of monocytes. J. Immunol. August 1; 151(3):1280-9 (1993)
Rolland J, O'Hehir R. Immunotherapy of allergy: anergy, deletion, and immune deviation. Curr Opin Immunol. December; 10(6):640-5 (1998)
Royer B, Varadaradjalou S, Saas P, Guillosson J J, Kantelip J P, Arock M. Inhibition of IgE-induced activation of human mast cells by IL-10. Clin Exp Allergy. May; 31(5):694-704 (2001)
Savolainen J, Laaksonen K, Rantio-Lehtimaki A, Terho E O. Increased expression of allergen-induced in vitro interleukin-10 and interleukin-18 mRNA in peripheral blood mononuclear cells of allergic rhinitis patients after specific immunotherapy. Clin Exp Allergy. March; 34(3):413-9 (2004)
Schramm G, Kahlert H, Suck R, Weber B, Stuwe H T, Muller W D, Bufe A, Becker W M, Schlaak M W, Jager L, Cromwell O, Fiebig H. “Allergen engineering”: variants of the timothy grass pollen allergen Phl p 5b with reduced IgE-binding capacity but conserved T cell reactivity. J. Immunol. February 15; 162(4):2406-14 (1999)
Secrist H, Chelen C J, Wen Y, Marshall J D, Umetsu D T. Allergen immunotherapy decreases interleukin 4 production in CD4+ T cells from allergic individuals. J Exp Med. December 1; 178(6):2123-30 (1993)
Swoboda I, De Weerd N, Bhalla P L, Niederberger V, Sperr W R, Valent P, Kahlert H, Fiebig H, Verdino P, Keller W, Ebner C, Spitzauer S, Valenta R, Singh M B. Mutants of the major ryegrass pollen allergen, Lol p 5, with reduced IgE-binding capacity: candidates for grass pollen-specific immunotherapy. Eur J. Immunol. January; 32(1):270-80 (2002)
Takai T, Yokota T, Yasue M, Nishiyama C, Yuuki T, Mori A, Okudaira H, Okumura Y. Engineering of the major house dust mite allergen Der f 2 for allergen-specific immunotherapy. Nat. Biotechnol. August; 15(8):754-8 (1997)
Takanaski S, Nonaka R, Xing Z. O'Byrne P, Dolovich J, Jordana M. Interleukin 10 inhibits lipopolysaccharide-induced survival and cytokine production by human peripheral blood eosinophils. J Exp Med. August 1; 180(2):711-5 (1994)
van Hage-Hamsten M, Kronqvist M, Zetterstrom O, Johansson E, Niederberger V, Vrtala S, Gronlund H, Gronneberg R, Valenta R. Skin test evaluation of genetically engineered hypoallergenic derivatives of the major birch pollen allergen, Bet v 1: results obtained with a mix of two recombinant Bet v 1 fragments and recombinant Bet v 1 trimer in a Swedish population before the birch pollen season. J Allergy Clin Immunol. November; 104(5):969-77 (1999)
Van Ree R, Van Leeuwen W A, Dieges P H, Van Wijk R G, De Jong N, Brewczyski P Z, Kroon A M, Schilte P P, Tan K Y, Simon-Licht I F, Roberts A M, Stapel S O, Aalberse R C. Measurement of IgE antibodies against purified grass pollen allergens (Lol p 1, 2, 3 and 5) during immunotherapy. Clin Exp Allergy. January; 27(1):68-74 (1997)
Vrtala S, Akdis C A, Budak F, Akdis M, Blaser K, Kraft D, Valenta R. T cell epitope-containing hypoallergenic recombinant fragments of the major birch pollen allergen, Bet v 1, induce blocking antibodies. J Immunol. December 1; 165(11):6653-9 (2000)
Walker S M, G B Pajno M T Lima, D R Wilson, and S R Durham. Grass pollen immunotherapy for seasonal rhinitis and asthma: a randomized, controlled trial. J. Allergy Clin. Immunol. 107:87 (2001)
Wilson D R, Irani A M, Walker S M, Jacobson M R, Mackay I S, Schwartz L B, Durham S R. Grass pollen immunotherapy inhibits seasonal increases in basophils and eosinophils in the nasal epithelium. Clin Exp Allergy. November; 31(11):1705-13 (2001)
Zeiler T, Taivainen A, Rytkonen M, Rautiainen J, Karjalainen H, Mantyjarvi R, Tuomisto L, Virtanen T. Recombinant allergen fragments as candidate preparations for allergen immunotherapy. J Allergy Clin Immunol. December; 100(6 Pt 1):721-7 (1997)

1-29. (canceled)

30. An isolated polypeptide, derivative or fragment thereof, selected from the group consisting of:
(a) a polypeptide, derivative, isoform or fragment thereof, comprising a non-helical mutant of at least one Group 5 allergen wherein the Group 5 allergen is from mite; and
(b) the polypeptide, derivative, isoform or fragment thereof of (a), comprising at least one amino acid substitution, addition, deletion, or at least one chemical modification, and wherein the polypeptide exhibits the same or further reduced IgE reactivity than that of the polypeptide of (a) in subjects allergic to at least one Group 5 allergen, wherein the Group 5 allergen is from mite.

31. The polypeptide according to claim 30, wherein the Group 5 allergen is selected from the group consisting of Blo t 5, Der p 5, Der f 5, and Der m 5.

32. The polypeptide according to claim 30 wherein the Group 5 allergen is Blo t 5 allergen.

33. The polypeptide, derivative, isoform or fragment according to claim 30, wherein the polypeptide comprises an amino acid sequence of at least 60% homology with SEQ ID NO:33.

34. The polypeptide, derivative, isoform or fragment according to claim 30 comprising an amino acid sequence of SEQ ID NO:33.

35. The polypeptide according to claim 30, wherein the polypeptide comprises at least one of the amino acid substitutions corresponding to Leu57, Arg58, Arg74, Glu75, Glu79, Lys92, Lys94, Glu98, Gln102, Asp105, Lys106 of SEQ ID NO:33.

36. The polypeptide according to claim 30, wherein the Group 5 allergen is from at least one organism selected from the group consisting of Blomia genus, Dermatophagoides genus, Euoglyphus genus, Glycyphagus genus, Lepidoglyphus genus, Acarus genus and Tyrophagus genus.

37. An isolated polypeptide, derivative, isoform or fragment thereof, comprising at least one amino acid sequence selected from the group consisting of SEQ ID NO:17, SEQ ID NO:19, SEQ ID NO:21, SEQ ID NO:23, SEQ ID NO:25, SEQ ID NO:27, SEQ ID NO:29, SEQ ID NO:31, and SEQ ID NO:33.

38. An isolated nucleic acid molecule selected from the group consisting of SEQ ID NO:16, SEQ ID NO:18, SEQ ID NO:20, SEQ ID NO:22, SEQ ID NO:24, SEQ ID NO:26, SEQ ID NO:28, SEQ ID NO:30, and SEQ ID NO:32.

39. A vector comprising at least one nucleic acid molecule according to claim 38.

40. A host cell comprising the nucleic acid molecule according to claim 38.

41. A method for producing a polypeptide, the method comprising culturing the host cell according to claim 40 in a medium and isolating the polypeptide from the culture.

42. An isolated oligonucleotide comprising at least one nucleic acid sequence selected from the group consisting of SEQ ID NO:1 to SEQ ID NO:13 and SEQ ID NO:15.

43. A pharmaceutical composition comprising an effective amount of at least one polypeptide according to claim 30, and optionally further comprising at least one pharmaceutically acceptable carrier, diluent, adjuvant, excipients, or a combination thereof.

44. A vaccine comprising at least one polypeptide according to claim 30.

45. The vaccine according to claim 44, further comprising at least one adjuvant.

46. A method of treating at least one allergy in a subject, the method comprising administrating to the subject a polypeptide, derivative, isoform or fragment according to claim 30.

47. The method according to claim 46, wherein the allergy is a Type 1 allergy.

48. The method according to claim 46 where the administration is local, subcutaneous, intravenal, topical to a tissue locus, parenteral or oral.

49. A method according to claim 46 wherein the treatment is for specific allergen immunotherapy (SIT).

50. An isolated antibody, wherein the antibody specifically binds to at least one polypeptide according to claim 30.

51. A kit comprising at least one polypeptide according to claim 30.

52. A kit comprising at least one pharmaceutical composition according to claim 43.

53. A kit comprising at least one vaccine according to claim 44.

54. A diagnostic assay for assessing relevance, safety or outcome of therapy of a subject comprising at least one polypeptide according to claim 30 or at least one antibody, wherein the antibody specifically binds to the polypeptide, or at least one IgE-containing sample of the subject is mixed with the polypeptide or antibody and assessed for the level of IgE reactivity.


SEQUENCE LISTING (33 sequences)

SEQ ID NO: 1 - DNA, 39 bp, ARTIFICIAL
  Primer A-F, Table 1
gctatcgaaaagggacatcaagaattgctttacttgcaa

SEQ ID NO: 2 - DNA, 39 bp, ARTIFICIAL
  Primer A-R, Table 1
ttgcaagtaaagcaattcttgatgtcccttttcgatagc

SEQ ID NO: 3 - DNA, 60 bp, ARTIFICIAL
  Primer B-F, Table 1
atgatcgaaggagcccaaggagctttgcgcgaagaattgaaggaaactgatcttaacatt

SEQ ID NO: 4 - DNA, 57 bp, ARTIFICIAL
  Primer B-F, Table 1
tccttgggctccttcgatcatggcgcaaacaacatcaaggcgtcgaatgattttctc

SEQ ID NO: 5 - DNA, 45 bp, ARTIFICIAL
  Primer C1-F, Table 1
actctcagcaagatcttgcttcagaaattggatgaaaccgaacaa

SEQ ID NO: 6 - DNA, 36 bp, ARTIFICIAL
  Primer C1-R,Table 1
aagcaagatcttgctgagagttttagcctcttcgta

SEQ ID NO: 7 - DNA, 60 bp, ARTIFICIAL
  Primer C2-F, Table 1
aaagctcaaactctcagcgaaatcttgcttgaagatttgaagaaaaccgaacaaaaagtg

SEQ ID NO: 8 - DNA, 33 bp, ARTIFICIAL
  Primer C2-R, Table 1
ttcgctgagagtttgagctttttcgtagttgaa

SEQ ID NO: 9 - DNA, 57 bp, ARTIFICIAL
  Primer L/B-R, Table 1
tccttgggctccttcgatcatggcgcaaacaacatcaaggcgtagaatgattttctc

SEQ ID NO: 10 - DNA, 48 bp, ARTIFICIAL
  Primer C1C2-F, Table 1
ctcagcgaaatcttgcttcaggatttggataaaaccgaacaaaaagtg

SEQ ID NO: 11 - DNA, 45 bp, ARTIFICIAL
  Primer C1C2-R, Table 1
ctgaagcaagatttcgctgagagttttagctttttcgtagttgaa

SEQ ID NO: 12 - DNA, 23 bp, ARTIFICIAL
  Primer G4Fa, Table 1
gtgtttcgagaattgcatatagt

SEQ ID NO: 13 - DNA, 22 bp, ARTIFICIAL
  Primer G4Ra, Table 1
acctgacgtctaagaaaccatt

SEQ ID NO: 14 - DNA, 23 bp, ARTIFICIAL
  Primer pGEX-F, Table 1
gggctggcaagccacgtttggtg

SEQ ID NO: 15 - DNA, 23 bp, ARTIFICIAL
  Primer pGEX-R, Table 1
ccgggagctgcatgtgtcagagg

SEQ ID NO: 16 - DNA, 354 bp, ARTIFICIAL
  Blo t 5 mutant 1 (M1) - nucleic acid sequence
caagagcacaagccaaagaaggatgatttccgaaacgaattcgatcacttgttgatcgaacaggcaaaccatgctatcgaaaagggacatcaagaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaagaggctcaaactctcagcaagatcttgcttaaggatttgaaggaaaccgaacaaaaagtgaaggatattcaaacccaataa

SEQ ID NO: 17 - protein, 117 aa, ARTIFICIAL
  Synthetic Construct
QEHKPKKDDFRNEFDHLLIEQANHAIEKGHQELLYLQHQLDELNENKSKELQEKIIRELDVVCAMIEGAQGALERELKRTDLNILERFNYEEAQTLSKILLKDLKETEQKVKDIQTQ

SEQ ID NO: 18 - DNA, 354 bp, ARTIFICIAL
  Blo t 5 mutant 2 (M2) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgacgccttgatgttgtttgcgccatgatcgaaggagcccaaggagctttgcgcgaagaattgaaggaaactgatcttaacattttggaacgattcaactacgaagaggctcaaactctcagcaagatcttgcttaaggatttgaaggaaaccgaacaaaaagtgaaggatattcaaacccaataa

SEQ ID NO: 19 - protein, 117 aa, ARTIFICIAL
  Synthetic Construct
QEHKPKKDDFRNEFDHLLIEQANHAIEKGEHQLLYLQHQLDELNENKSKELQEKIIRRLDVVCAMIEGAQGALREELKETDLNILERFNYEEAQTLSKILLKDLKETEQKVKDIQTQ

SEQ ID NO: 20 - DNA, 354 bp, ARTIFICIAL
  Blo t 5 mutant 3 (M3) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaagaggctaaaactctcagcaagatcttgcttcagaaattggatgaaaccgaacaaaaagtgaaggatattcaaacccaataa

SEQ ID NO: 21 - protein, 117 aa, ARTIFICIAL
  Synthetic Construct
QEHKPKKDDFRNEFDHLLIEQANHAIEKGEHQLLYLQHQLDELNENKSKELQEKIIRELDVVCAMIEGAQGALERELKRTDLNILERFNYEEAKTLSKILLQKLDETEQKVKDIQTQ

SEQ ID NO: 22 - DNA, 354 bp, ARTIFICIAL
  Blo t 5 mutant 4 (M4) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaaaaagctcaaactctcagcgaaatcttgcttgaagatttgaagaaaaccgaacaaaaagtgaaggatattcaaacccaataa

SEQ ID NO: 23 - protein, 117 aa, ARTIFICIAL
  Synthetic Construct
QEHKPKKDDFRNEFDHLLIEQANHAIEKGEHQLLYLQHQLDELNENKSKELQEKIIRELDVVCAMIEGAQGALERELKRTDLNILERFNYEKAQTLSEILLEDLKKTEQKVKDIQTQ

SEQ ID NO: 24 - DNA, 354 bp, ARTIFICIAL
  Blo t 5 mutant 5 (M5) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattctacgccttgatgttgtttgcgccatgatcgaaggagcccaaggagctttgcgcgaagaattgaaggaaactgatcttaacattttggaacgattcaactacgaagaggctcaaactctcagcaagatcttgcttaaggatttgaaggaaaccgaacaaaaagtgaaggatattcaaacccaataa

SEQ ID NO: 25 - protein, 117 aa, ARTIFICIAL
  Synthetic Construct
QEHKPKKDDFRNEFDHLLIEQANHAIEKGEHQLLYLQHQLDELNENKSKELQEKIILRLDVVCAMIEGAQGALREELKETDLNILERFNYEEAQTLSKILLKDLKETEQKVKDIQTQ

SEQ ID NO: 26 - DNA, 354 bp, ARTIFICIAL
  Blo t 5 mutant 6 (M6) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaaaaagctaaaactctcagcgaaatcttgcttcaggatttggataaaaccgaacaaaaagtgaaggatattcaaacccaataa

SEQ ID NO: 27 - protein, 117 aa, ARTIFICIAL
  Synthetic Construct
QEHKPKKDDFRNEFDHLLIEQANHAIEKGEHQLLYLQHQLDELNENKSKELQEKIIRELDVVCAMIEGAQGALERELKRTDLNILERFNYEKAKTLSEILLQDLDKTEQKVKDIQTQ

SEQ ID NO: 28 - DNA, 354 bp, ARTIFICIAL
  Blo t 5 mutant 7 (M7) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattctagaacttgatgttgtttgcgccatgatcgaaggagcccaaggagctttggaacgtgaattgaagcgaactgatcttaacattttggaacgattcaactacgaaaaagctaaaactctcagcgaaatcttgcttcaggatttggataaaaccgaacaaaaagtgaaggatattcaaacccaataa

SEQ ID NO: 29 - protein, 117 aa, ARTIFICIAL
  Synthetic Construct
QEHKPKKDDFRNEFDHLLIEQANHAIEKGEHQLLYLQHQLDELNENKSKELQEKIILELDVVCAMIEGAQGALERELKRTDLNILERFNYEKAKTLSEILLQDLDKTEQKVKDIQTQ

SEQ ID NO: 30 - DNA, 354 bp, ARTIFICIAL
  Blo t 5 mutant 8 (M8) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgacgccttgatgttgtttgcgccatgatcgaaggagcccaaggagctttgcgcgaagaattgaaggaaactgatcttaacattttggaacgattcaactacgaaaaagctaaaactctcagcgaaatcttgcttcaggatttggataaaaccgaacaaaaagtgaaggatattcaaacccaataa

SEQ ID NO: 31 - protein, 117 aa, ARTIFICIAL
  Synthetic Construct
QEHKPKKDDFRNEFDHLLIEQANHAIEKGEHQLLYLQHQLDELNENKSKELQEKIIRRLDVVCAMIEGAQGALREELKETDLNILERFNYEKAKTLSEILLQDLDKTEQKVKDIQTQ

SEQ ID NO: 32 - DNA, 354 bp, ARTIFICIAL
  Blo t 5 mutant 9 (M9) - nucleic acid sequence
caagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattctacgccttgatgttgtttgcgccatgatcgaaggagcccaaggagctttgcgcgaagaattgaaggaaactgatcttaacattttggaacgattcaactacgaaaaagctaaaactctcagcgaaatcttgcttcaggatttggataaaaccgaacaaaaagtgaaggatattcaaacccaataa

SEQ ID NO: 33 - protein, 117 aa, ARTIFICIAL
  Synthetic Construct
QEHKPKKDDFRNEFDHLLIEQANHAIEKGEHQLLYLQHQLDELNENKSKELQEKIILRLDVVCAMIEGAQGALREELKETDLNILERFNYEKAKTLSEILLQDLDKTEQKVKDIQTQ